(19) United States
(12) Patent Application Publication
(10) Pub. No.: US 2024/0361025 A1
(43) Pub. Date: Oct. 31, 2024

(54) AIR PURIFIER

(71) Applicant: TOTAL ASSIST CO., LTD., Nagoya-shi, Aichi (JP)

(72) Inventor: Masanobu ICHIKAWA, Nagoya-shi (JP)

(73) Assignee: TOTAL ASSIST CO., LTD., Nagoya-shi, Aichi (JP)

(21) Appl. No.: 18/287,846

(22) PCT Filed: Apr. 28, 2022
(86) PCT No.: PCT/JP2022/019235

§ 371 Date: Oct. 20, 2023

(30) Foreign Application Priority Data
May 11, 2021 (JP) 2021-080312
Jul. 26, 2021 (JP) 2021-121169
Dec. 28, 2021 (JP) 2021-214235

(51) Int. Cl. F24F 8/15; F24F 8/22; F24F 11/72

(52) U.S. Cl. CPC F24F 8/15; F24F 8/22; F24F 11/72

(57) ABSTRACT

An air purifier includes: an air passage; an antimicrobial function part placed in the air passage and configured to inactivate pathogens; and a staying time regulation part that regulates the time for which the air passing through the air passage stays within the air passage. This air purifier may further include a blower fan for causing air to pass through the air passage, or an air-flow resistance member that impedes the passage of air within the air passage. The staying time regulation part is configured to iteratively switch between a stagnating state, in which the staying time of air in the air passage is long, and an air-flowing state, in which the staying time of air therein is short.

### TECHNICAL FIELD

The present disclosure relates to an air purifier for purifying the air passing therethrough and, in particular, relates to an air purifier configured to inactivate pathogens, such as viruses.

### BACKGROUND ART

As a conventional device having a function of purifying the air passing therethrough, there is an “air cleaner” disclosed in Patent Document 1. The air cleaner disclosed in this document includes various filters in a housing. These filters trap or catch airborne minute foreign substances, such as powder dust, and further are expected to trap pathogens, such as viruses, as well.

### RELATED ART DOCUMENTS

### Patent Documents

Patent Document 1: Japanese Utility Model Registration No. 3226941

### SUMMARY OF INVENTION

### Problems to be Solved by the Invention

The aforementioned conventional art has the following problems. The filters are apt to clog, resulting in deterioration of air permeability. Even if the air cleaner is activated while the filters are clogged, those filters cannot sufficiently exert the function of purifying the air. If a coarse filter is used, it is less likely to clog, but such a filter can hardly trap microscopic particles, such as pathogens. Therefore, even if the filter is exposed to ultraviolet light or the like, actually, the filter does not sufficiently exhibit the expected effect of inactivating the pathogens.

The present disclosure has been made to address the above problems of the conventional art and specifically has a purpose to provide an air purifier capable of reliably inactivating pathogens contained in the air that passes through the air purifier.

### Means of Solving the Problems

One aspect of the present disclosure provides an air purifier including an air passage; an antimicrobial function part placed in the air passage configured to inactivate pathogens; and a staying-time adjusting unit configured to adjust a staying time of air for which the air stays in the air passage in passing through the air passage. In the air purifier configured as above, the staying time of air in the air passage is adjusted by the staying-time adjusting unit to make a long-staying-time state and a short-staying-time state. The long-staying-time state is a state where air stagnates in the air passage. In this state, the antimicrobial function part acts on the air stagnating in the air passage to inactivate pathogens. This reduces the concentration of pathogens in the air stagnating in the air passage. When the staying-time adjusting unit adjusts the staying time to make the short-staying-time state, the air with reduced concentration of pathogens is allowed to be discharged from the air passage. Thus, the air in a room where the air purifier is disposed turns to a state with fewer active pathogens.

Preferably, the air purifier configured as above includes a blower fan for causing air to pass through the air passage, and the staying-time adjusting unit is used as an air-blowing power regulating unit for regulating air-blowing power of the blower fan. When the air-blowing power of the blower fan is set high, the staying time of air becomes short. In contrast, when the air-blowing power is set low, the staying time of air becomes long.

Alternatively, the air purifier configured as above may include an air-flow resistance member for impeding air from passing through the air passage, and a staying-time adjusting unit may be used as an opening and closing operation unit for changing air-flow resistance of the air-flow resistance member. When the airflow resistance of the air-flow resistance member is set low, the staying time of air becomes short. In contrast, when the airflow resistance is set high, the staying time of air becomes long.

In the air purifier including the blower fan or the air-flow resistance member, furthermore, the staying-time adjusting unit is configured to iteratively switch between a stagnating state where the staying time of air in the air passage is long and an air-flowing state where the staying time of air in the air passage is short. When this switching operation is continued, the air in the room where the air purifier is disposed can be maintained with fewer active pathogens.

The air purifier configured to perform the iteratively switching may further include a plurality of sets of the air passages and a plurality of the antimicrobial function parts, in which the staying-time adjusting unit is configured to: adjust the staying time of air in each of the air passages individually, and perform cycle control in which some of the air passages are brought into the stagnating state and the remaining air passages are brought into the air-flowing state, while the air passage targeted for the stagnating state is switched in sequence. Depending on the number of sets of air passages and antimicrobial function parts, the air purifier can address a large capacity, and also any one of the air passages can be in the air-flowing state at all times.

In the air purifier configured to perform the cycle control, furthermore, the staying-time adjusting unit is also preferably configured to: group the pairs of the air passages and the antimicrobial function parts into a plurality of groups; and perform the cycle control on each of the groups separately according to the staying time different for each group. This configuration can also flexibly address various types of pathogens.

An air purifier in another aspect of the present disclosure includes an air passage; an ultraviolet lamp or an electric heating plate, placed in the air passage and configured to inactivate pathogens; and a staying-state switching unit configured to switch between an air-flowing state where air is allowed to pass through the air passage and a stagnating state where air is stationary in the air passage. In the air purifier configured as above, the staying state of air in the air passage is switched by the staying-state switching unit to make a long-staying-time state and a short-staying-time state. The long-staying-time state is a state where air stagnates in the air passage. In this state, the ultrasonic lamp or the electric heating plate acts on the air stagnating in the air passage to inactivate pathogens. This reduces the concentration of pathogens in the air stagnating in the air passage. When the staying state is switched to the short-staying-time state, the air with reduced concentration of pathogens is allowed to be discharged from the air passage. This allows the air in a room where the air purifier is disposed to be changed to a state with fewer active pathogens.

Alternatively, the above-configured air purifier includes an opening and closing valve placed in the air passage, and the staying-state switching unit is configured to switch the opening and closing valve between an open state and a closed state. When the opening and closing valve is switched to the open state, the staying time of air becomes short. When the opening and closing valve is switched to the closed state, the staying time of air becomes long. The ultraviolet lamp or electric heating plate is turned on at least in the stagnating state.

In the air purifier in any one of the aforementioned configurations, preferably, the air purifier includes a blower fan for causing air to pass through the air passage, and the staying-state switching unit is used as an air-blowing power regulating unit that switches the blower fan between an air-blowing state and a stop state. When the air-blowing power of the blower fan is set high, the staying time of air becomes short. When the blower fan is stopped, the staying time of air becomes long.

In the air purifier configured to include the blower fan or the opening and closing valve, preferably, the staying-state switching unit is also configured to iteratively switch between the air-flowing state and the stagnating state. When this switching operation is continued, the air in a room where the air purifier is disposed can be maintained with fewer active pathogens.

### Effects of the Invention

According to the present disclosure, an air purifier is provided capable of reliably inactivating pathogens contained in the air in passing through the air purifier.

### BRIEF DESCRIPTION OF THE DRAWINGS

FIG. 1 is a configuration view of an air purifier in a first basic embodiment;

FIG. 2 is a cross-sectional view showing the inside of an antimicrobial function part (First example);

FIG. 3 is a cross-sectional view showing the inside of an antimicrobial function part (Second example);

FIG. 4 is a cross-sectional view showing the inside of an antimicrobial function part (Third example);

FIG. 5 is a cross-sectional view showing the inside of an antimicrobial function part (Fourth example);

FIG. 6 is a configuration view of a modified example of the air purifier in the first basic embodiment;

FIG. 7 is a configuration view of an air purifier in a second basic embodiment;

FIG. 8 is a schematic diagram showing a usage state of the air purifier illustrated in FIG. 7;

FIG. 9 is a configuration view of an air purifier in an applied configuration;

FIG. 10 is a timing chart of an operation example of the air purifier illustrated in FIG. 9;

FIG. 11 is a configuration diagram showing a modified example of performing cycle control per group; and

FIG. 12 is a configuration view of a modified example of the air purifier illustrated in FIG. 1.

### MODE FOR CARRYING OUT THE INVENTION

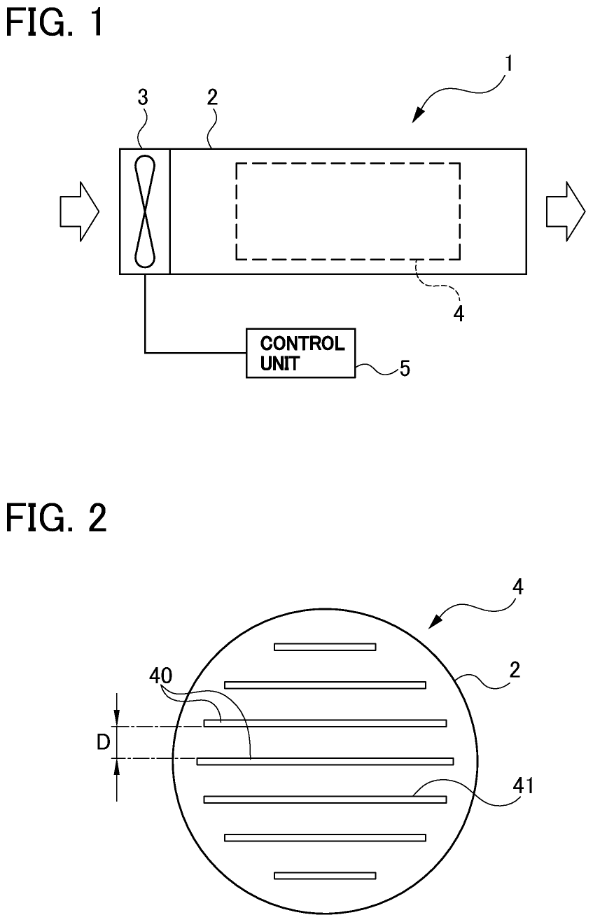

A detailed description of embodiments of this disclosure will now be given referring to the accompanying drawings. Basic embodiments will be described first. An air purifier 1 in the first basic embodiment is configured as shown in FIG. 1. The air purifier 1 in FIG. 1 includes a tubular member 2. This tubular member 2 is hollow and allows the air to pass therethrough. In the air purifier 1, the internal space of the tubular member 2 is used as an air passage. A blower fan 3 is installed at an entrance of the tubular member 2. An antimicrobial function part 4 is built in the tubular member 2. The blower fan 3 is operated by a control unit 5. That is, the control unit 5 is an air-blowing power regulating unit for controlling the air-blowing power of the blower fan 3.

The antimicrobial function part 4 has the function of inactivating pathogens, such as viruses and bacteria, contained in the air. Among many types of the antimicrobial function part 4, the present embodiment adopts a sterilization plate type. As shown in a cross-sectional view in FIG. 2, the antimicrobial function part 4 is provided with sterilizing plates 40 arranged in the tubular member 2. In the configuration example in FIG. 2, a plurality of sterilizing plates 40 are arranged horizontally. At least the upper surface of each sterilizing plate 40 is a sterilizing plane 41, which is coated with an agent for inactivating pathogens. As the agent to be applied to the surface of each sterilizing plate 40 to form the sterilizing plane 41, for example, there are an agent made of titanium dioxide, silver, and calcium phosphate hydroxide (e.g., an agent containing “Cerami.D.A” (registered trademark)), or an ammonium salt-based agent (e.g., an agent named “GlossWell”).

The air purifier 1 configured as above is iteratively switched between an air-blowing state in which air-blowing is executed by the blower fan 3 and an air-blowing stop state in which the air-blowing is stopped. This operation of the blower fan 3 is controlled by the control unit 5. During the air-blowing by the blower fan 3, the air is caused to flow into the tubular member 2 through its inlet and flow out from the tubular member 2 through its outlet. This state is an air-flowing state.

While the blower fan 3 is stopped blowing air, the air in the tubular member 2 is stationary, i.e., is in a so-called stagnating state. In this stationary air, airborne pathogens fall down due to the action of gravity, and land on the sterilizing planes 41. The landed pathogens are then inactivated by the effect of the sterilizing planes 41. The longer the air-blowing stop time, the fewer the number of pathogens remaining floating in the air. In addition, the narrower the interval (D in FIG. 2) between the sterilizing plates 40 in the vertical direction, the shorter time it takes for the pathogens to land on the sterilizing planes 41. Consequently, the narrower the interval D, the more likely the effect of inactivating pathogens is exerted even during a short air-blowing stop time. The pathogens inactivated once on the sterilizing planes 41 are still inactive even if they come free from the sterilizing planes 41 by the air blown afterward by the restarted blower fan 3.

The airborne pathogens fall down at average falling velocities different between in the droplet form and in the non-droplet form, which are assumed to be about 300 to 800 mm/sec in the form of droplet and about 0.6 to 15 mm/sec in the form of nucleus. In general, for the interval D of about 10 to 20 mm, most of airborne pathogens land within 30 seconds. For the air-blowing stop time of about 60 seconds, the number of pathogens that remain in the air decreases to 0.1% or less of the original number of pathogens. From this it is conceived that one stop time in the control of intermittently operating the blower fan 3 by the control unit 5 is set to about 60 to 180 seconds. When the target type of pathogens is determined in advance, the one stop time may be set depending on the nature of the pathogens. Even if a new type of pathogen emerges, the above setting can be adapted to the target new-type pathogen if its nature becomes revealed.

In contrast, one air-blowing time may be basically determined based on the relationship between the air-blowing power of the blower fan 3 and the internal volume of the tubular member 2, more strictly, the effective volume of the antimicrobial function part 4. Assuming that the air-blowing power of the blower fan is P (m³/min) and the internal volume of the tubular member 2 is V (m³), the time T (min) required to replace the air in the tubular member 2 by the air blown by the blower fan is given by V/P. For example, when the air-blowing power P is 1 (m³/min) and the internal volume V is 1 (m³), the time T is 1 (min). For the same air-blowing power P, if the internal volume V is twice, the time T is also twice. The time T calculated based on the air-blowing power P and the internal volume V serves as a standard of the one air-blowing time.

However, the one air-blowing time does not need to be set so strictly. Even if it is longer than the time obtained by V/P as described above, the effects can be produced to some extent. If the one air-blowing time is long, especially at the end stage thereof, the number of pathogens in the discharged air will not so largely change from the original number of airborne pathogens. However, pathogens are continuously trapped by the sterilizing planes 41 and thus the total number of pathogens in the room air is still reduced just by the number of the trapped pathogens. Therefore, if no pathogens newly enter into the room air, the number of pathogens in the room air can be gradually reduced by iterative operation of the air purifier 1. Even if a certain amount of pathogens newly enter into the room air, the iterative operations of the air purifier 1 can reduce the number of pathogens in the room air compared to when the air purifier 1 is not used.

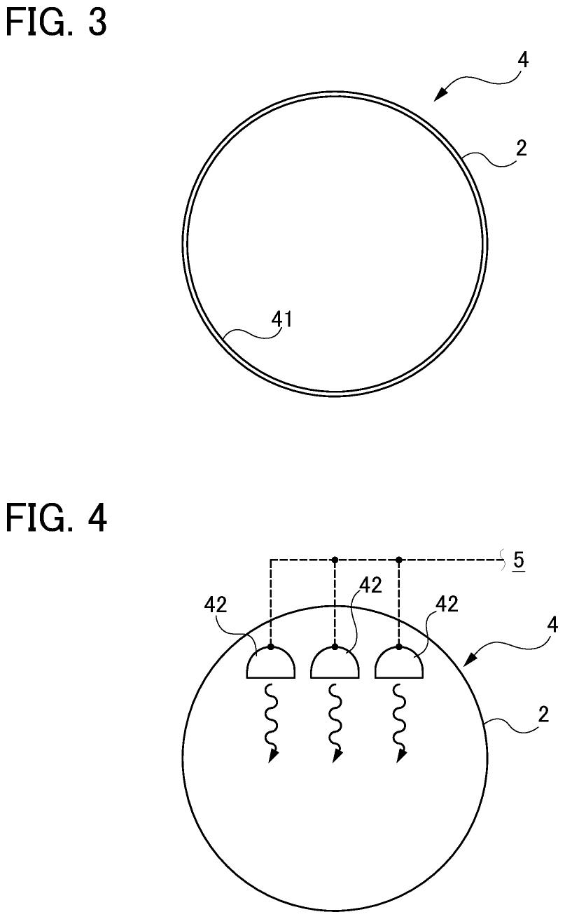

The configuration of the antimicrobial function part 4 in the air purifier 1 is not limited to that shown in FIG. 2. For example, as shown in FIG. 3, the inner surface of the tubular member 2 itself may be formed as the sterilizing plane 41, instead of arranging the sterilizing plates 40. The thus configured antimicrobial function part 4 needs a longer air-blowing stop time than that shown in FIG. 2 but can provide the same function as that in FIG. 2 by iteratively switching between air-blow and stop. In the antimicrobial function part 4 shown in FIG. 3, the entire inner surface of the tubular member 2 may be formed as the sterilizing plane 41 or only a lower part of the inner surface may be formed as the sterilizing plane 41.

When only the lower part is formed as the sterilizing plane 41, the tubular member 2 may be designed with a non-circular cross-section or with an outer surface marked with an indication so that the top and bottom of the tubular member 2 can be identified even from its external appearance. For the tubular member 2 in FIG. 2, similarly, it is more preferable that the top and bottom of the tubular member 2 can be identified even from its external appearance.

Still another example of the configuration of the antimicrobial function part 4 will be shown in FIG. 4 illustrating an example of utilizing the sterilizing action of ultraviolet light, not of the sterilizing plane 41. In the configuration example in FIG. 4, the antimicrobial function part 4 is provided with ultraviolet lamps 42 in the tubular member 2. In this configuration example, the ultraviolet light emitted from the ultraviolet lamps 42 inactivates the pathogens inside the tubular member 2. Since the ultraviolet light directly activates airborne pathogens, this configuration does not require consideration of the landing of falling pathogens, as in the configuration examples in FIGS. 2 and 3.

However, even in the configuration example in FIG. 4, the blower fan 3 is still iteratively switched between the air-blowing state and the stop state. This is because not all the pathogens in the tubular member 2 are inactivated at the moment when the ultraviolet lamps 42 are turned on for irradiation of ultraviolet light. When this irradiation continues for a certain amount of time while air blowing is stopped, it is possible to significantly reduce the number of active pathogens. Even in the configuration example in FIG. 4, desirably, one stop time is set to about 30 to 60 seconds.

The irradiation of ultraviolet light only has to be performed during the air-blowing stop state. In this case, turning on and off of the ultraviolet lamps 42 is opposite to turning on and off of the blower fan 3. For this purpose, the turning on and off of the ultraviolet lamps 42 may also be controlled by the control unit 5. However, even if the ultraviolet lamps 42 are left ever on, no particular adverse effects occur. In that case, the ultraviolet lamps 42 do not have to be controlled by the control unit 5.

When the ultraviolet light and photocatalyst are to be used together, the contact efficiency between the ultraviolet light and the photocatalyst can also be enhanced. In this case, photocatalytic plates are arranged in multiple stages, as with the sterilizing plates 40 in FIG. 2, and the ultraviolet lamps 42 are placed between the stages. In this configuration, the photocatalyst exposed to the ultraviolet light decomposes and inactivates the pathogens. Thus, the pathogens in the air fall down onto the photocatalytic plates and are inactivated thereon, as in the case of FIG. 2.

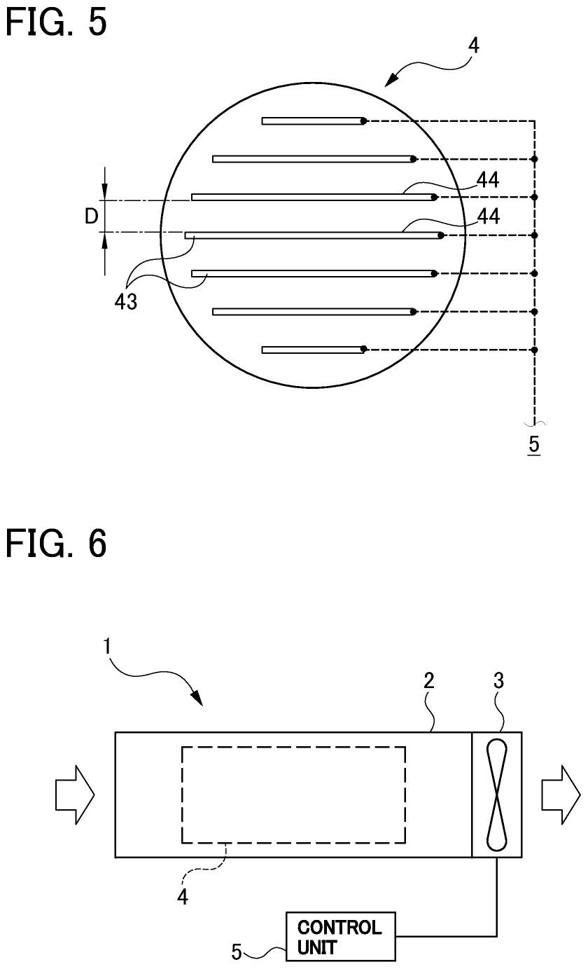

FIG. 5 shows another configuration, which is similar to FIG. 2, but in which electric heating plates 43 are arranged in place of the sterilizing plates 40. At least an upper surface of each electric heating plate 43 is formed as a heating surface 44. This heating surface 44 is a surface on which electric heating wires are laid out, generating Joule heat when they are applied with electric current, thereby inactivating pathogens. The interval D between the electric heating plates 43 in the configuration example in FIG. 5 may be considered as with the interval D in the configuration example in FIG. 2. In the configuration example in FIG. 5, similarly, the air-blowing state and the stop state are iteratively switched.

The heating surfaces 44 only have to be turned on during the air-blowing stop state. However, even if the heating surfaces 44 are left ever on, no particular adverse effects occur. To turn on the heating surfaces 44 only during the air-blowing stop state, they may be controlled by the control unit 5, as with the ultraviolet lamps 42 in the configuration example of FIG. 4. The electric current of the heating surfaces 44 may be set enough to warm the heating surfaces 44 to about 100° C., or even to a higher temperature. For example, even the temperature of 200° C. corresponding to a high temperature of a clothes iron, or higher, 400° C. or 1000° C., is adoptable as long as the surrounding structures are resistant to such high temperatures. Each current-on time, i.e., each air-blowing stop time, may be set to about 1 minute.

The heating surface 44 may be configured with a rubber heater or a ceramic heater as well as commonplace electric wires. Even the antimicrobial function part 4 including the heating surface 44 can be designed with the cross-section as shown in FIG. 3. The antimicrobial function part 4 may be selected from various types, such as a mist spraying type and an electric adsorbing type, in addition to the aforementioned types. Two or more of those types may be combined to constitute the antimicrobial function part 4.

The air purifier 1 configured as above can produce the excellent air purifying effect by the inactivating function of the antimicrobial function part 4 and the iterative operations of the blower fan 3. During a period of the air-blowing stop state, air is stagnant in the tubular member 2. During this period, the number of pathogens in the stagnant air is significantly reduced by the function of the antimicrobial function part 4. The thus purified air flows out from the tubular member 2 through its outlet during a period of the air-blowing state. Accordingly, the air in a room where the air purifier 1 is disposed is purified. As the air-blowing state continues for about the aforementioned time T, a situation may occur in which air flowing in the tubular member 2 through its inlet flows out therefrom through its outlet as it is. Thus, when or before such a situation comes about, the air purifier 1 is shifted to the air-blowing stop state again. By repetition of the above operations, the room air is kept clean with few pathogens.

In other words, in the air-blowing stop state, the staying time of air in the tubular member 2 is very long. In contrast, in the air-blowing state, the staying time of air is shorter than that in the air-blowing stop state. From this point of view, the control unit 5 that adjusts the air-blowing power of the blower fan 3 also serves as a staying-time adjusting unit for adjusting the staying time of air in the tubular member 2.

In the air purifier 1 described above, the tubular member 2 with a larger inner diameter allows air to flow at a higher air flow rate. The inner diameter of the tubular member 2 can be selected according to the dimensions of a room where the air purifier 1 will be disposed. Even when the antimicrobial function part 4 configured as shown in FIG. 2 or 5 has a tubular member 2 with a large inner diameter, the interval D can be set at an appropriate value simply by increasing the number of the sterilizing plates 40 or the electric heating plates 43. Furthermore, the longer the length of the tubular member 2, the larger the internal volume of the tubular member 2. Thus, if the length of the antimicrobial function part 4 is set long according to the length of the tubular member 2, a large amount of clean air to be discharged in one air-blowing period can be obtained.

Most of conventional devices would be configured to trap airborne pathogens with filters, which are for example made of non-woven fabrics, while allowing air to constantly pass therethrough. However, such a configuration is not so effective in actually inactivating pathogens for the following reasons. Among pathogens, viruses are as very microscopic as 300 nm or less. Therefore, such filters as used in ordinary air cleaners cannot trap the viruses. Since usual filters have no function of inactivating pathogens, even if viruses are trapped by the filters, the viruses may be discharged as they remain active after coming free from the filters.

Some very fine-meshed filters are considered to be able to trap viruses to a certain extent. However, in actual air, larger substances than viruses, such as powder dust, pollen, and bacteria, are also floating. Those larger-sized particles will be trapped by the filters earlier than viruses. This causes clogging and deteriorated air-permeability, as described in the section titled “PROBLEMS TO BE SOLVED BY THE INVENTION”.

In contrast, the air purifier 1 in the present embodiment can reliably discharge out only the air with the significantly reduced number of pathogens from the tubular member 2 through its outlet. This is because this air purifier 1 does not utilize a filter in order to trap pathogens. Even if the air purifier 1 used herein is a combination type of ultraviolet light and photocatalyst, the photocatalyst is not utilized as a filter in the present embodiment and thus no clogging problem occurs.

The utilization of the air purifier 1 in the present embodiment in a room provides the following advantages. The air purifier 1 in the present embodiment does not affect the air pressure in the room because the amount of air flowing into the air purifier 1 is equal to the amount of air discharged therefrom. This does not increase the load on an air conditioner. Rather, the utilization of the air purifier 1 reduces the need for ventilation, leading to a reduced load on the air conditioner. For the antimicrobial function part 4 of an electrically heating type, it generates heat, which may accordingly cause a load on the air conditioner, but this will not cause a problem during heating seasons. Even if there is a negative pressure tent for medical use in the room, the air purifier 1 in the present embodiment will not induce air leakage from the negative pressure tent.

A modified example of the entire configuration of the air purifier 1 in the first basic embodiment will be described below. In the air purifier 1, the position of the blower fan 3 is not limited to that shown in FIG. 1. Instead of placing the blower fan 3 at the inlet of the tubular member 2, it may be placed at the outlet as shown in FIG. 6. Blower fans 3 may be placed one at each of the inlet and the outlet. The blower fan 3 may be placed at some midway point in the tubular member 2. In this case, the antimicrobial function part 4 may be placed either on an upstream side or a downstream side of the blower fan 3, or alternatively, on both sides.

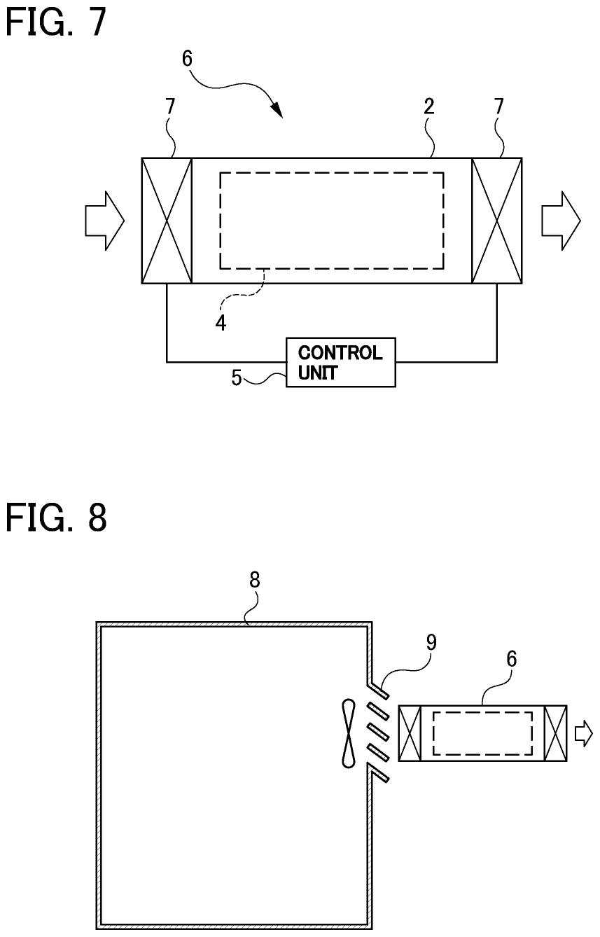

Next, an air purifier 6 in a second basic embodiment shown in FIG. 7 will be described below. The air purifier 6 in FIG. 7 is identical to the air purifier 1 in FIG. 1 excepting that opening and closing valves 7 are mounted in the inlet and the outlet of the tubular member 2 instead of the blower fan or fans 3. The opening and closing valves 7 are each controlled by the control unit 5. That is, the control unit 5 is an opening and closing operation unit. The opening and closing valve 7 may be placed only in one of the inlet and the outlet. The opening and closing valve 7 may be placed at some midway point in the tubular member 2. In this case, the antimicrobial function part 4 may be placed either on an upstream aide or a downstream side of the opening and closing valve 7, or alternatively, on both sides. The tubular member 2 and the antimicrobial function part 4 are identical to those in the air purifier 1 in the first basic embodiment.

Unlike the air purifier 1 in FIG. 1, the air purifier 6 itself in FIG. 7 does not have the function of actively causing air to flow in the tubular member 2. The opening and closing valve 7 is an air-flow resistance member that impedes the air from passing through the tubular member 2. The opening and closing valve 7 in a closed state provides a stagnating state with high airflow resistance, whereas the opening and closing valve 7 in an open state provides an air-flowing state with low airflow resistance. Specifically, the control unit 5 also serves as a staying-time adjusting unit for adjusting the staying time of air in the tubular member 2 by switching the opening and closing valve 7 to the open state or the closed state. The air purifier 6 in FIG. 7 is intended to be used together with some other device 8. This other device 8 is a device with an exhaust port 9, such as an air conditioner, and may be any existing device.

In FIG. 8, the air purifier 6 is placed with its inlet facing the exhaust port 9 of the other device 8. In this orientation, when the opening and closing valves 7 of the air purifier 6 are both opened, the air discharged from the exhaust port 9 are thus allowed to pass through the inside of the tubular member 2. When the opening and closing valves 7 are closed, the air discharged from the exhaust port 9 does not enter in the tubular member 2 and hence the air in the tubular member 2 becomes stagnant. Specifically, the open state of the opening and closing valves 7 correspond to the air-blowing state in FIG. 1, while the closed state of the opening and closing valves 7 corresponds to the air-blowing stop state in FIG. 1. By iteratively switching the open state period and the closed state period of the opening and closing valves 7 at appropriate frequencies, the air purifier 6 can thus exhibit the air purifying function as with the air purifier 1 in FIG. 1. As an alternative, the air purifier 6 may be placed such that the outlet of the tubular member 2 faces a device that sucks air, such as a circulator, instead of being oriented as shown in FIG. 8.

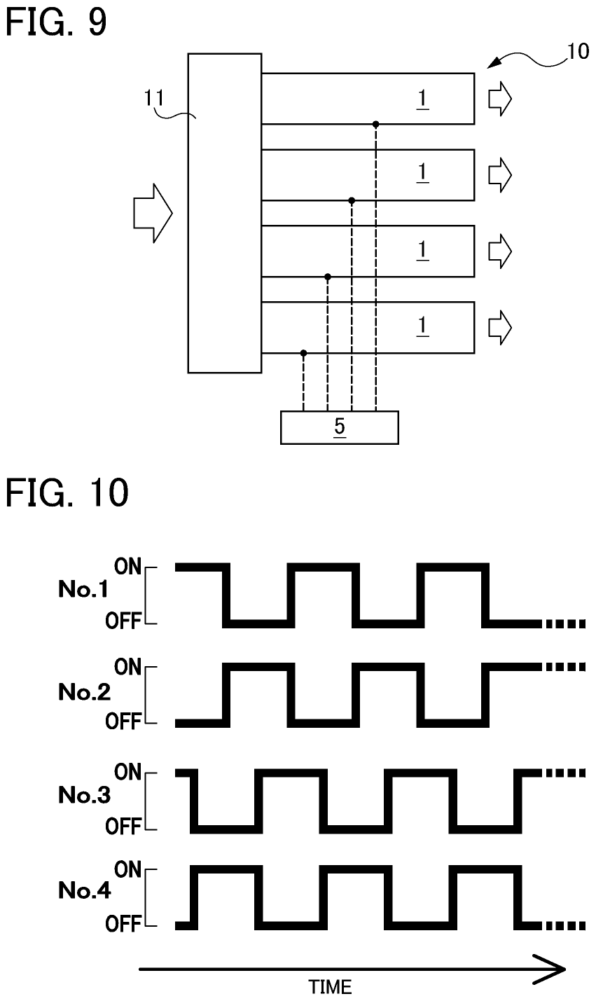

An air purifier in an applied embodiment will be described below. FIG. 9 shows an air purifier 10 composed of a plurality of air purifiers 1 shown in FIG. 1. In this air purifier 10, the air purifiers 1 are mounted on a manifold 11. In this configuration, air flows into the manifold 11 first and therefrom is distributed to flow in the air purifiers 1. The air purifiers 1 are each controlled by the control unit 5. The control unit 5 individually controls the control units 5 of the air purifiers 1.

The air purifier 10 in FIG. 9 can operate to switch the air purifiers 1 in sequence, as shown in FIG. 10, for example. In other words, some of the air purifiers 1 are brought into the air-blowing stop state (the stagnating state) and the remaining air purifiers 1 are brought into the air-blowing state (the air-flowing state), while the air purifiers 1 targeted for the air-blowing stop state are shifted in sequence. These operations are of course controlled by the control unit 5. This is called a cycle control.

Under the above control, the air purifier 10 as a whole can always discharge air after purifying even when each air purifier 1 is intermittently operated. By this mechanism, one air-blowing stop time in each air purifier 1 may also be set longer than one air-blowing time. The longer one air-blowing stop time enables the antimicrobial function part 4 to reliably inactivate pathogens. FIGS. 9 and 10 merely show one example. The total number of air purifiers 1 and the number of air purifiers 1 that will be simultaneously turned on can be selected arbitrarily. The arrangement of the air purifiers 1 is not limited to the parallel arrangement as shown in FIG. 9, but also may be a radial arrangement. The air purifier 10 may be composed of the air purifiers 6 in FIG. 7 instead of the air purifiers 1 in FIG. 1. The air purifier 10 may also be composed of a plurality of air purifiers 6 in FIG. 7 and a single blower fan 3.

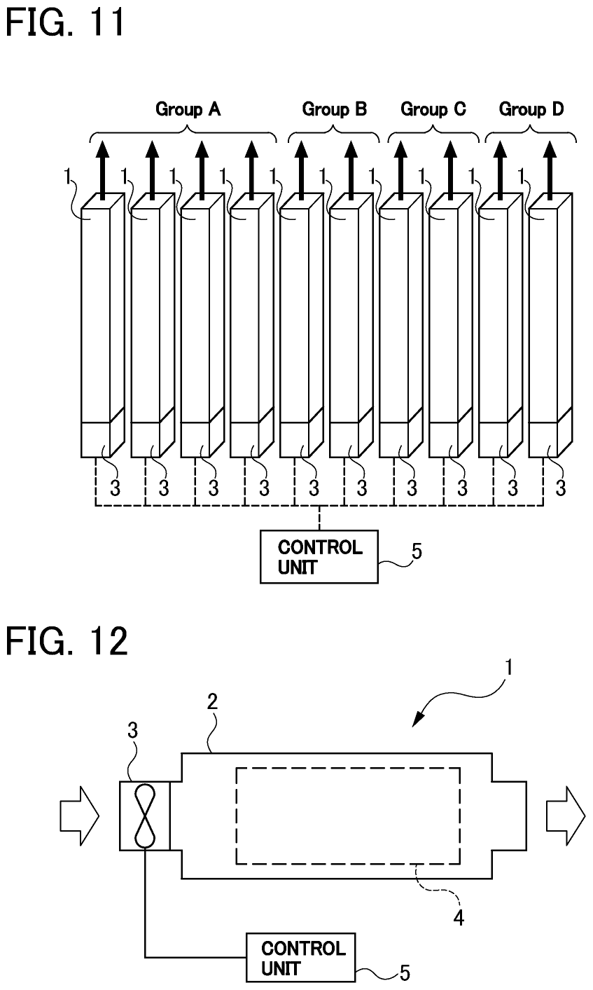

FIG. 11 shows a configuration example for the cycle control to be performed per group. In this example, ten air purifiers 1 are grouped into four groups A, B, C, and D. Each group includes a plurality of air purifiers 1. Thus, the cycle control can be performed separately for each group. This operation is also controlled by the control unit 5. To be specific, for example, the following operations are conceivable.

| | | |
|---|---|---|
| (Group A) | Target: | First type of virus (tolerance: 6 seconds), |
| | | Second type of virus (tolerance: 8 seconds) |
| | Air-blowing | 10 seconds |
| | stop time: | |
| (Group B) | Target: | Third type of virus (tolerance of: 10 seconds) |
| | Air-blowing | 15 seconds |
| | stop time: | |
| (Group C) | Target: | Bacteria (tolerance: 20 seconds) |
| | Air-blowing | 20 seconds |
| | stop time: | |
| (Group D) | Target: | Fourth type of virus (tolerance: 30 seconds) |
| | Air-blowing | 30 seconds |
| | stop time: | |

When the air staying time is set different for each group as above, the air purifiers 1 can address various types of pathogens. For each group, the staying time has to be set equal to or somewhat longer than the tolerance time (i.e., the time required to inactivate most of the pathogens in each air purifier 1) according to the type of pathogens assumed as the target. The air-blowing time may be set appropriately according to the capacity of the air purifiers 1 and the air-blowing rate of the blower fans 3. Even if new or mutated types of pathogens emerge, the air purifiers 1 can also address them by setting the staying time according to the tolerance time of the new pathogens if it is ascertained. The number of groups and the number of air purifiers 1 in each group may be set arbitrarily. The number of air purifiers 1 may be different from group to group as shown in FIG. 11.

As described above in detail, the air purifier 1, 6 in the present embodiments includes the antimicrobial function part 4 in the tubular member 2 and configured to switch between the air-blowing state and the air-blowing stop state by operations of the blower fan 3 or opening and closing valves 7. With the above configuration, the air purifier 1, 6 is switched between the air-blowing state and the air-blowing stop state. In this way, the air in the tubular member 2 in the air-blowing stop state is made clean with less pathogens, and such a clean air is discharged in the air-blowing state and simultaneously new air before purifying is taken in the tubular member 2.

Thus, the air purifier 1, 6 can be realized capable of reliably inactivating the pathogens contained in the air passing therethrough, not trapping the pathogens with a filter. By iterating the air-blowing state and the air-blowing stop state, the air purifier 1, 6 can reduce the concentration of airborne pathogens in the room where the air purifier 1, 6 is disposed. The plurality of air purifiers 1 or 6 may be combined to constantly discharge purified air.

The use of the air purifier 1, 6, 10 can also provide the effect of maintaining the room in a state with airflow. This airflow in the room can suppress the pathogens from falling down in the room, sticking to tables and human bodies. This means that the air purifier 1, 6, 10 in the present embodiments is very advantageous especially in commercial facilities, etc., and in mid-summer or mid-winter, when even ventilation with outside air is desirably avoided. This is because the air purifier 1, 6, 10 can reduce pathogens without increasing the operation load of the air conditioner.

The air purifier 1, 6, 10 in the present embodiments is also advantageous compared to ordinal air purifiers configured to trap foreign substances by a filter. This is because the filter cannot inactivate pathogens even if they trap the pathogens, and then those trapped pathogens, remaining active, will inevitably separate from the filter after a while.

The foregoing embodiments are mere examples and give no limitation to the present disclosure. The present disclosure may be embodied in other specific forms without departing from the essential characteristics thereof. In addition to several modified examples described in the foregoing embodiment, for example, the tubular member 2 may be designed so that the inlet and the outlet are narrower than a middle portion therebetween, as shown in FIG. 12. The same may apply to the configuration in FIG. 7. The tubular member 2 of the air purifier 1, 6 is not limited to the illustrated straight shape, but may be curved in some midpoint. The tubular member 2 may also include a branch junction or a confluence junction in some midpoint. If the branch or confluence portion is provided, it may be located either upstream or downstream of the antimicrobial function part 4, but desirably there is no path that extends from the inlet to the outlet but does not pass through the antimicrobial function part 4. An air purifier may be provided with both the blower fan 3 and the opening and closing valve 7 in the tubular member 2.

The opening and closing valves 7 of the air purifier 6 may be configured to be able to open partially, not limited to the fully open position and the fully closed position. In this case, the air-blowing stop state of the air purifier 6 does not necessarily require the opening and closing valve 7 to be fully closed. This opening degree of the opening and closing valve 7 has only to be smaller than the opening degree in the air-blowing state and need a longer time for air to pass through the tubular member 2 than required to purify the air through the antimicrobial function part 4. Similarly for the blower fan 3 of the air purifier 1, the blower fan 3 may be rotated at a very low speed even in the air-blowing stop state.

The air purifier 10 may be composed of a combination of the air purifier(s) 1 and the air purifier(s) 6. The antimicrobial function parts 4 may be different in type for each air purifier 1 or each air purifier 6. The length of the tubular member 2 and the cycle of switching between the air-blowing stop state and the air-blowing state may be different for each air purifier 1 or each air purifier 6.

### REFERENCE SIGNS LIST

1 Air purifier
2 Tubular member
3 Blower fan
4 Antimicrobial function unit
5 Control unit
6 Air purifier
7 Opening and closing valve
10 Air purifier
40 Sterilizing plate
41 Sterilizing plane
42 Ultraviolet lamp
43 Electric heating plate
44 Heating surface

1. An air purifier comprising:
an air passage;
an antimicrobial function part placed in the air passage and configured to inactivate pathogens;
a blower fan for causing air to pass through the air passage; and
a staying-time adjusting unit configured to adjust a staying time of air for which the air stays in the air passage in passing through the air passage,
wherein the staying-time adjusting unit is
used as an air-blowing power regulating unit for regulating air-blowing power of the blower fan, and
configured to iteratively switch between a stagnating state where the staying time of air in the air passage is long and an air-flowing state where the staying time of air in the air passage is short, the air purifier includes a plurality of sets of the air passages and the antimicrobial function parts, and
the staying-time adjusting unit is configured to:
adjust the staying time of air in each of the air passages individually, and
perform cycle control in which some of the air passages are brought into the stagnating state and the remaining air passages are brought into the air-flowing state, while the air passage targeted for the stagnating state is shifted in sequence.

2. An air purifier comprising:
an air passage;
an antimicrobial function part placed in the air passage and configured to inactivate pathogens;
an air-flow resistance member for impeding air from passing through the air passage; and
a staying-time adjusting unit configured to adjust a staying time of air for which the air stays in the air passage in passing through the air passage,
wherein the staying-time adjusting unit is
used as an opening and closing operation unit for changing air-flow resistance of the air-flow resistance member, and
configured to iteratively switch between a stagnating state where the staying time of air in the air passage is long and an air-flowing state where the staying time of air in the air passage is short,
the air purifier includes a plurality of sets of the air passages and the antimicrobial function parts, and
the staying-time adjusting unit is configured to:
adjust the staying time of air in each of the air passages individually, and
perform cycle control in which some of the air passages are brought into the stagnating state and the remaining air passages are brought into the air-flowing state, while the air passage targeted for the stagnating state is shifted in sequence.

3. The air purifier according to claim 1, wherein
the staying-time adjusting unit is configured to:
group the pairs of the air passages and the antimicrobial function parts into a plurality of groups; and
perform the cycle control on each of the groups separately according to the staying time different for each group.

4-7. (canceled)

8. The air purifier according to claim 2, wherein
the staying-time adjusting unit is configured to:
group the pairs of the air passages and the antimicrobial function parts into a plurality of groups; and
perform the cycle control on each of the groups separately according to the staying time different for each group.
